(19) United States
(12) Patent Application Publication
(10) Pub. No.: US 2010/0241161 A1
(43) Pub. Date: Sep. 23, 2010

(54) SURGICAL FASTENERS AND DEVICES FOR SURGICAL FASTENING

(75) Inventor: Gilad Heftman, Kibbutz Ein Gev (IL)

Correspondence Address: THE NATH LAW GROUP, 112 South West Street, Alexandria, VA, 22314, US

(73) Assignee: I.B.I Israel Biomedical Innovations Ltd., Kibbutz Haogen (IL)

(21) Appl. No.: 12/733,170

(22) PCT Filed: Aug. 17, 2008
(86) PCT No.: PCT/IL2008/001130

§ 371 Date: May 7, 2010

Related U.S. Application Data
(60) Provisional application No. 60/935,513, filed on Aug. 16, 2007.

(51) Int. Cl. A61B 17/08

(52) U.S. Cl. 606/213

(57) ABSTRACT

Disclosed is a surgical fastener having a first element, or crown, defining an axis of the fastener. Each prong of the fastener has a protuberance at one end that is received in a depression around an edge of the first element and rotatable in the depression and has a tip at a second end. In an undeployed configuration, each prong passes through a slot of the second element and the tip of each prong is at a first distance from the axis. In a deployed configuration, each prong passes through the prong's slot of the second element, and the tip of each prong is at a second distance from the axis that is greater than the first distance. Also disclosed is a system for surgical fastening including one or more surgical fasteners as disclosed herein and a surgical fastening device configured to deploy the fasteners.

### FIELD OF THE INVENTION

This invention relates to surgical fasteners and to surgical fastening devices.

### BACKGROUND OF THE INVENTION

Surgical anchors are used instead of surgical suturing, which is often both time consuming and inconvenient, in order to join two tissue locations. A surgeon can often use a stapling apparatus to implant an anchor into a body tissue and thus accomplish in a few seconds, what would take a much longer time to suture. A surgical anchor is used, for example in inguinal hernia surgery to fasten polypropylene mesh to the abdominal wall in order to reinforce the abdominal wall.

Conventional surgical fasteners have been in the form of ordinary metal staples, which are bent by the delivery apparatus to join together body tissues. These staples comprise a pair of legs or prongs joined together at one end by a crown that may be straight or arcuate. During deployment of the staple, the prongs are inserted into a tissue and are then made to bend inwards towards.

At present, there are a variety of surgical fasteners and fastening devices available for endoscopic or open procedures, to attach tissues together, or to attach a mesh patch to a tissue. One such surgical fastener is a surgical stapler, or clip applicator. In this stapler, a plurality or stack of unformed staples are contained within a cartridge and are sequentially advanced or fed within the instrument by a spring mechanism. A secondary feeding mechanism is employed to separate the distal most staple from the stack, and to feed the distal most stapler into the staple closing mechanism. Such mechanisms are found in U.S. Pat. Nos. 5,470,010, and 5,582,616.

In some applications, the body tissue is accessible from two opposite direction so that an anvil may be used to deform the legs of a staple after having passed through the body tissue. In applications where access to the tissue is from only one direction, an anvil may be used to deform the crown of a conventional staple so that the legs project towards each other in the body tissue so as to hold the staple in the tissue.

Another stapler mechanism, used mostly for mesh attachment to tissue does not use an anvil. Instead, a fastener comprising a helical wire is screwed or rotated into a tissue, in order to join tissues to affix a polypropylene or similar material mesh or other patch to the tissue together. Instruments and fasteners of this type are found in U.S. Pat. No. 5,582,616, U.S. Pat. No. 5,810,882, and U.S. Pat. No. 5,830,221. Another type of fastener that does not need an anvil applies fasteners made from a shape memory alloy such as Nitinol™. These fasteners are mainly used to fasten prosthetic material or artificial mesh to tissue.

These fasteners and fastening devices suffer from significant drawbacks especially when attaching fasteners to soft tissue. The strength of attachment of these devices depends mainly on the content and size of collagen fibers. Most soft tissue, such as subcutaneous tissue and fatty tissue surrounding internal organs, has few and slender collagen fibers and hence the attachment of the common art fasteners to such tissue is weaker than attachment to stronger tissues such as fascia or ligaments, which have more and larger collagen fibers.

### SUMMARY OF THE INVENTION

In its first aspect the invention provides a surgical fastener. The surgical fastener of the invention comprises two or more prongs each of which is connected to a first element by a hinge. The fastener is positioned at the site of a tissue surface where it is to be deployed. The fastener is then deployed by applying an extending force to the prongs so that the prongs splay radially outward from the first element by rotating at the hinges as they enter the body tissue so as to become embedded in the tissue. The fastener may be used to attach a graft or a mesh to a body tissue. In contrast to the prior art surgical anchors which penetrate the tissue entirely, in the case of the surgical fastener of the present invention, only the prongs of the fastener penetrate through the graft or mesh into the tissue, while the first element to which the prongs are connected attach the graft or mesh to the tissue surface without penetrating into it.

Thus, in one of its aspects, the invention provides a surgical fastener having an undeployed configuration and a deployed configuration, comprising:
(a) a first element defining an axis of the fastener having one or more depressions around an edge of the first element;
(b) two or more prongs, each prong having a protuberance at one end, the protuberance being received in one of the depressions of the first element and rotatable in the depression, and each prong having a tip at a second end; and
(c) a second element having one or more wells or holes and two or more slots extending from the well or hole to an edge of the second element;
   wherein in the undeployed configuration, each prong passes through a slot of the second element and the tip of each prong is at a first distance from the axis; and wherein, in the deployed configuration, each prong passes through the prong's slot of the second element, and the tip of each prong is at a second distance from the axis that is greater than the first distance.

The invention also provides a system for surgical fastening comprising:
(a) one or more surgical fasteners having an undeployed configuration and a deployed configuration, the surgical fasteners comprising:
   a first element defining an axis of the fastener having one or more depressions around an edge of the first element;
   two or more prongs, each prong having a protuberance at one end, the protuberance being received in one of the depressions of the first element and rotatable in the depression, and each prong having a tip at a second end; and
   a second element having one or more wells or holes and two or more slots extending from the well or hole to an edge of the second element;
wherein in the undeployed configuration, each prong passes through a slot of the second element and the tip of each prong is at a first distance from the axis; and wherein, in the deployed configuration, each prong passes through the prong's slot of the second element, and the tip of each prong is at a second distance from the axis that is greater than the first distance; and
(b) a fastening device configured to deploy one or more of the surgical fasteners.

The invention further provides a method for surgical fastening, comprising:
i) providing a system for surgical fastening, the system comprising comprising:
(a) one or more surgical fasteners having an undeployed configuration and a deployed configuration, the surgical fasteners comprising:
   a first element defining an axis of the fastener having one or more depressions around an edge of the first element;
   two or more prongs, each prong having a protuberance at one end, the protuberance being received in one of the depressions of the first element and rotatable in the depression, and each prong having a tip at a second end; and
   a second element having one or more wells or holes and two or more slots extending from the well or hole to an edge of the second element;
wherein in the undeployed configuration, each prong passes through a slot of the second element and the tip of each prong is at a first distance from the axis; and wherein, in the deployed configuration, each prong passes through the prong's slot of the second element, and the tip of each prong is at a second distance from the axis that is greater than the first distance; and
(b) a fastening device configured to deploy one or more of the surgical fasteners;
ii) deliverying at least a portion of the fastening device to a body site where a fastener is to be deployed; and
iii) using the fastening device to deploy fastener at the body site.

### BRIEF DESCRIPTION OF THE DRAWINGS

In order to understand the invention and to see how it may be carried out in practice, embodiments of the invention will now be described by way of non-limiting example only, with reference to the accompanying drawings, in which:

FIG. 1 shows a surgical fastener according to one embodiment of the invention in an un-deployed configuration in a side perspective view;

FIG. 2 shows the surgical fastener of FIG. 1 in a deployed configuration in a side perspective view;

FIG. 3a shows the surgical fastener FIG. 1 in the un-deployed configuration in longitudinal section;

FIG. 3b shows the surgical fastener FIG. 1 in the deployed configuration in longitudinal section;

FIG. 4 shows a stabilization device for stabilizing a fastener of the invention;

FIG. 5 shows an assembly comprising the undeployed fastener and the stabilization means;

FIG. 6 shows an enlarged view of the distal end of the shaft of a fastening device in accordance with this aspect of the invention;

FIG. 7 shows insertion of the assembly into a cartridge;

FIG. 8 shows mating of longitudinal recesses of the cartridge with longitudinal grippers situated at the distal end of a fastening device;

FIG. 9 shows a surgical fastener and the distal end of a surgical fastening device in accordance with another embodiment of the invention;

FIG. 10 shows locking of the fastener in its deployed configuration;

FIGS. 11 and 12 show use of a cartridge for mounting the fastener onto the distal end of a fastening device;

FIG. 13 shows separation of the crown and baseplate;

FIG. 14 shows a fastener of the invention provided with a thread or filament for removal of the fastener, when it is determined that the fastener has not been properly deployed;

FIG. 15 shows a surgical fastener in accordance with another embodiment of the invention;

FIGS. 16a and 16b show another surgical fastener in accordance with another embodiment of the invention;

FIG. 17 shows a stabilization device;

FIG. 18 shows a surgical fastener in accordance with another embodiment of the invention in which the prongs are organized into adjacent pairs, where the protrusions of the prongs of each pair are joined together; and

FIG. 19 shows deployment of the fastener of the invention.

### DESCRIPTION OF EMBODIMENTS

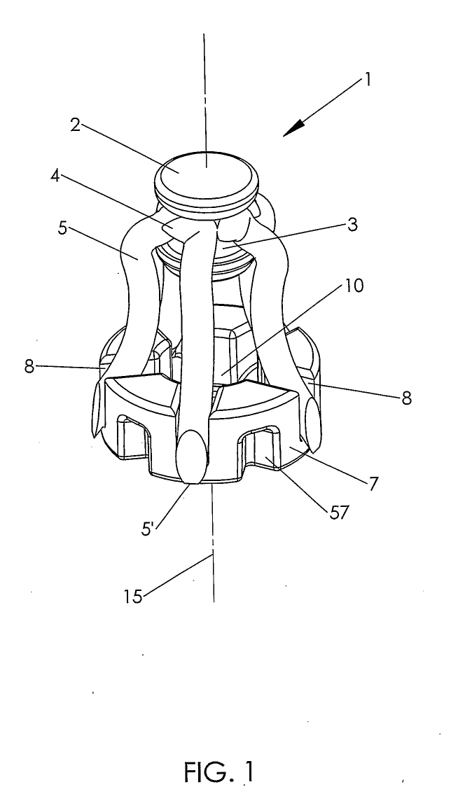

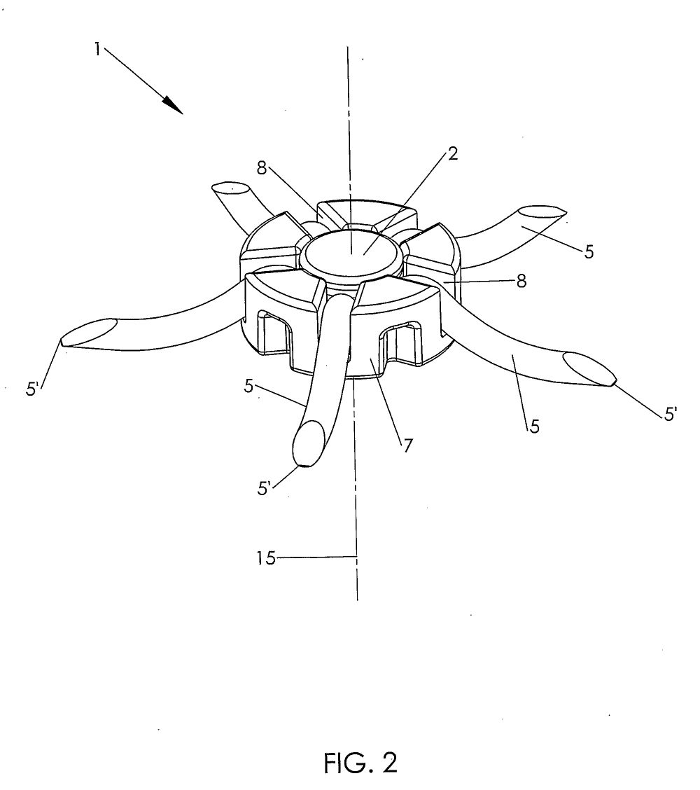

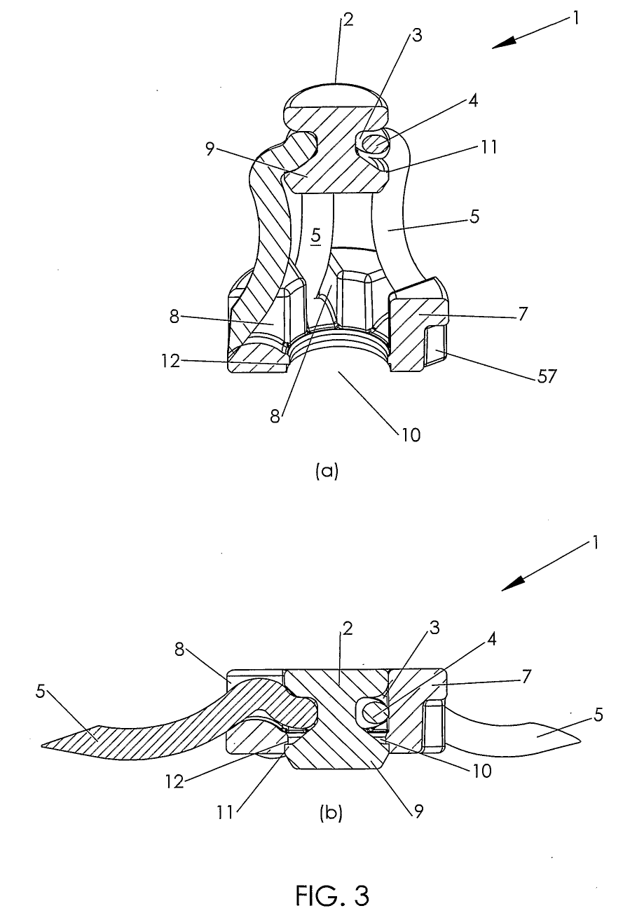

FIGS. 1, 2, and 3 show a surgical fastener 1 in accordance with one embodiment of the invention. The fastener 1 in its undeployed configuration is shown in a side perspective view in FIG. 1 and in a longitudinal section in FIG. 3a. The fastener 1 in its deployed configuration is shown in a side perspective view in FIG. 2 and in a longitudinal section in FIG. 3b. The fastener 1 comprises a first element or crown 2 that may have any shape, as required in any application. For example, the first element may have a shape such as a disk, or cylinder, a rectangular or polygonal shape, an irregularly shaped surface. The crown 2 defines a longitudinal axis 15 of the fastener 1.

Two or more prongs 5 extend from the crown 2. The crown 2 is provided with a depression 3 around an edge of the crown 2. The depression 3 is dimensioned to receive a protuberance 4 located at the base of each prong 5. The depression 3 and the corresponding protuberance 4 are dimensioned so that the protuberance 4 can rotate in the depression 3.

The fastener 1 may contain any number of prongs 5 that is at least two. The prongs may have any profile as required in any application, such as a rectangular profile, a round profile, an oval profile, a triangular profile, or an elliptical profile. The prongs may be straight or curved with constant or variable curvature. The prongs may have blunt tips, pointed tips or barbed tips, as required in any application.

The fastener 1 includes a second element or baseplate 7. The baseplate 7 has a central bore or well 10 from which extend two or more slots 8 to an edge of the baseplate 7. The prongs 5 and the slots 8 are dimensioned to allow each prong to pass through its slot either freely, or with some friction. The protuberance 4 of each prong 5 is dimensioned as to be unable to pass through the prong's slot. The second element may have any shape, as required in any application. For example, the second element may be a baseplate having a shape such as a disk, a rectangular or polygonal shaped surface, an irregularly shaped surface.

When the fastener 1 is in the undeployed configuration, the prong tips 5′ are closer to the axis 15 than when the fastener 1 is in the deployed configuration. In the deployed configuration, each prong passes through the prong's slot of the second element, and the tip of each prong is at a second distance from the axis that is greater than the first distance and the protuberance of each prong is located in one of the wells or bores of the second element. The protuberances are shaped and dimensioned so as to be prevented from passing through the prong's slot.

During deployment of the fastener 1, a fastening device is deployed that urges the first element 2 towards the baseplate 7, as described in detail below. As the prongs pass through the slots 8, the force applied to the prongs 5 by the slots 8 causes the prongs to rotate about the protuberances 4 and splay radially outward from the crown to attain the deployed configuration.

The fastener of the invention may be manufactured from any biocompatible, and preferentially biodegradable, materials such as but not limited to: PLA, PLGA, poly-caprolactone, polydiaxone, magnesium alloys or any combination of such materials.

The fastener of the invention is preferably locked in the deployed configuration in order to prevent unintended release of the fastener from the body tissue. As used herein, the term “locking” of the fastener refers to an engagement between components of the fastener that increases the force necessary to bring the extended prongs of the deployed fastener closer to the longitudinal axis of the fastener compared to situation in which such means are not provided. The result is that the fastener retains its deployed configuration despite the forces that act on it within the tissue and is better attached to the delicate soft tissue.

In a preferred embodiment, the locking is due to an engagement between a projection 9 (FIG. 3) of the crown 2 and the well or bore 10 in the baseplate 7. in the deployed configuration, the projection 9 of the crown is provided with a rim 11 that snap fits beneath a rim 12 provided within the well or bore 10 of the baseplate 7, as shown in FIG. 3b.

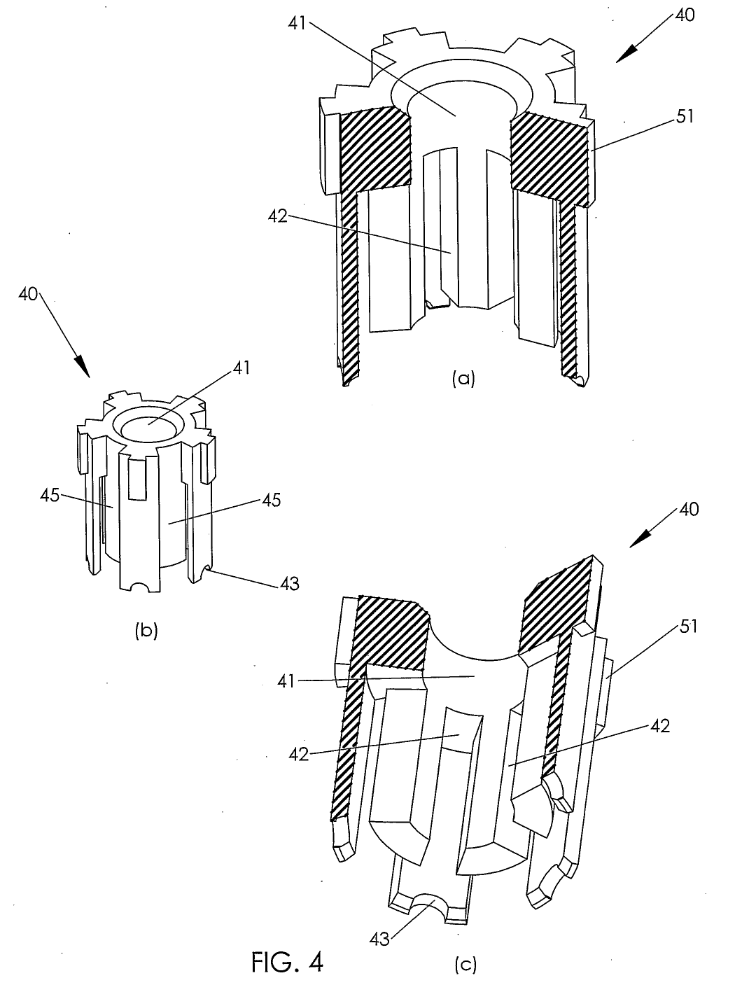

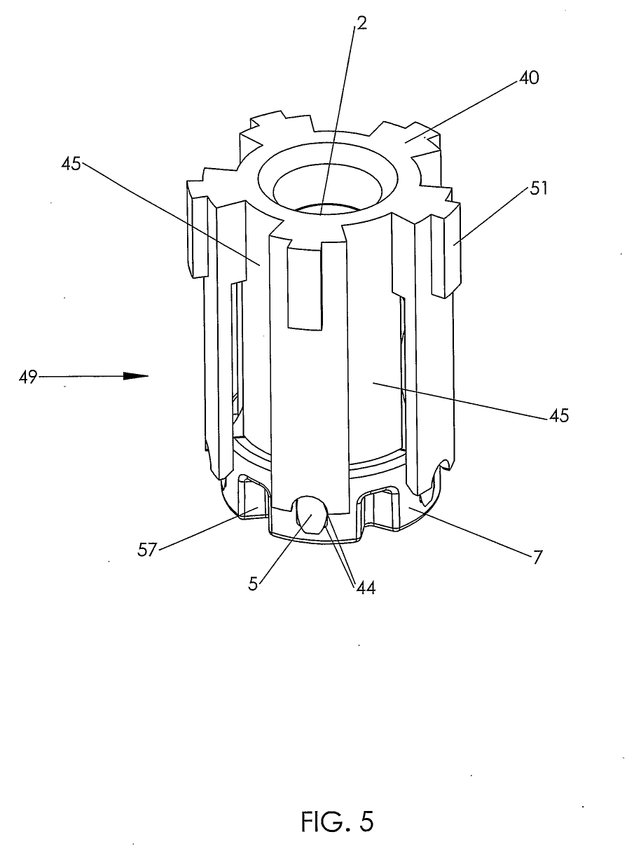

The protuberances 4 at the base of the prongs 5 are not necessarily retained in the depression 3 of the crown. In order to stabilize the fastener 1 in its undeployed configuration during loading of the fastener onto a fastening device, a stabilization device may be used. A stabilization device 40 for the fastener 1 is shown in FIG. 4 that may be used in cases where the fastener is unstable in its undeployed configuration. The stabilization device 40 is essentially cylindrical and comprises a central bore 41. A central bore 41 in the stabilization device 40 is configured to receive the undeployed fastener. The central bore 41 is provided with radially disposed recesses 42 for retaining the prongs 5 of the fastener along the longitudinal axis 15 of the undeployed fastener. These recesses 42 also prevent prongs 5 that have a curved shape from rotating along their long axis. FIG. 5 shows an assembly 49 comprising the undeployed fastener 1 and the mounted stabilization means 40. When the stabilization device 40 is mounted on the fastener 1, the protuberances 4 are prevented from disengaging from the depression 3. When the stabilization device 40 is mounted on the fastener 1 in the undeployed configuration of the fastener, notches 43 in the stabilization device 40 are aligned with the slots 8 of the baseplate to form channels 44. As explained below, the channels 44 house and guide the prongs 5 during splaying and deployment.

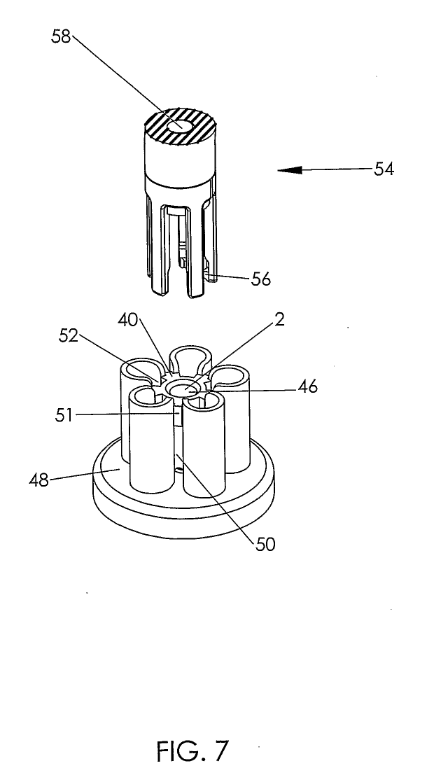

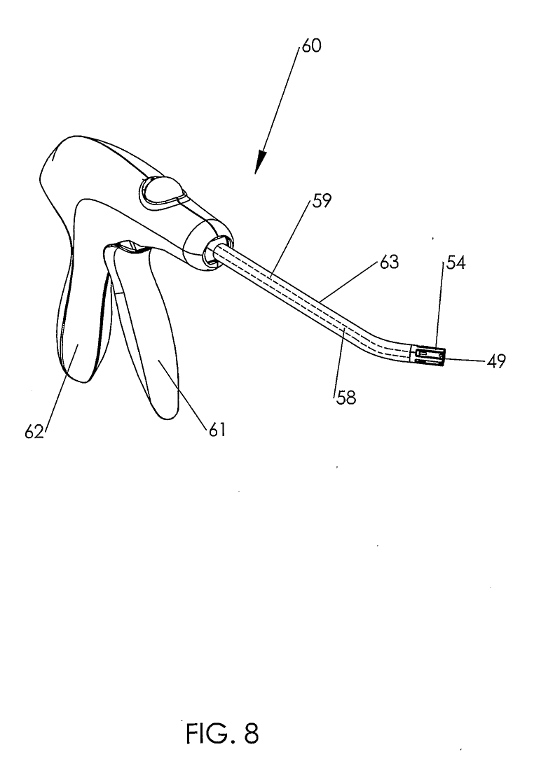

As shown in FIG. 7, the assembly 49 may be inserted into a cartridge 48 to prevent disengagement of the baseplate 7 during loading of the fastener 1 onto a fastening device. The cartridge 48 has a central bore 46 that is provided with longitudinal recesses 50 that mate with longitudinal projections 51 on the outer surface of the stabilization device 40. When the assembly 49 is introduced into the central bore of the cartridge 48, disengagement of the baseplate 7 from the prongs 5 of the undeployed fastener in the assembly is prevented. Additional longitudinal recesses 52 of the cartridge 48 mate with longitudinal grippers 47 situated at the distal end of a fastening device 60 (FIG. 8), as explained below.

In its second aspect the invention provides a surgical fastening device for deploying a surgical fastener of the invention, such as the fastener 1. As shown in FIG. 8, a preferred embodiment of the fastening device 60 of the invention is manufactured from biocompatible materials, such as biocompatible metallic or plastic materials, or a combination of them The fastening device 60 has a shaft 63 having a tip 54 configured to receive an assembly 49 comprising a fastener 1 to be deployed in a body tissue and a stabilization device 40. As explained below, the device is configured to compress the fastener by urging the crown 2 of the fastener towards the baseplate 7 as the prongs of the fastener splay radially outward from the first element by rotating at the protuberances 4. In use, is delivered to the body site where the fastener 1 is to be deployed. As the fastener is brought to its deployed configuration by the fastening device, the prongs splay out from the first element into the body tissue at that site. Once the fastener has attained its deployed configuration, the fastener is released from the fastening device.

The shaft of the deployment device may be straight or curved; rigid, semi-rigid or flexible. It may be flexible along its entire length or only at specific locations thus permitting manipulation of the shaft in narrow body spaces.

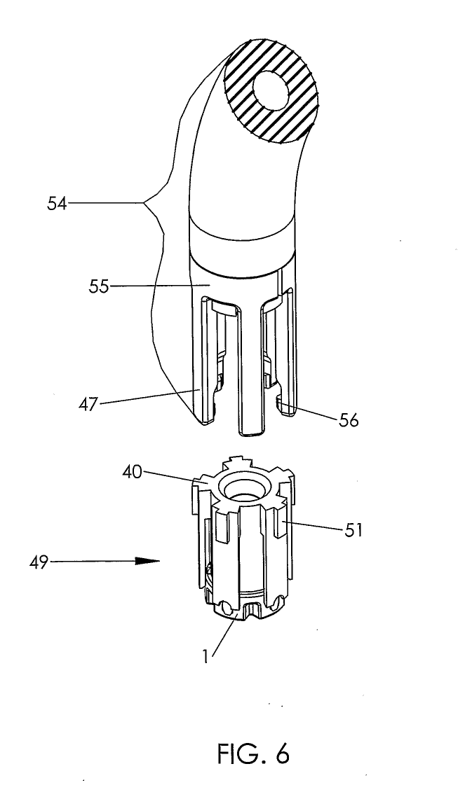

Referring now to FIG. 6, an enlarged view of the distal end 54 of the shaft of the fastening device 60 is shown. The shaft tip 54 of the fastening device 60 is provided at its distal rim 55 with longitudinal grippers 47 each of which is provided with a small projection 56 at its tip.

As shown in FIG. 7, in order to mount the assembly 49 when placed in the housing cartridge 48 onto the tip 54 of the fastening device 60, the grippers 47 of the fastening device tip 54 are introduced into the mating recesses 52 of the cartridge 48 and into the recesses 45 of the stabilization device 40. A small projection 56 at the tip of the grippers 47 engages small fitting recesses 57 of the baseplate 7 (see also FIG. 1). In this way, the undeployed fastener 1, in the assembly 49, is engaged in the fastening device tip 54 by the projections 56 of the grippers 47. The assembly 49 comprising the fastener 1 and the stabilizer 40, can then be removed from the cartridge 48, as shown in FIG. 8.

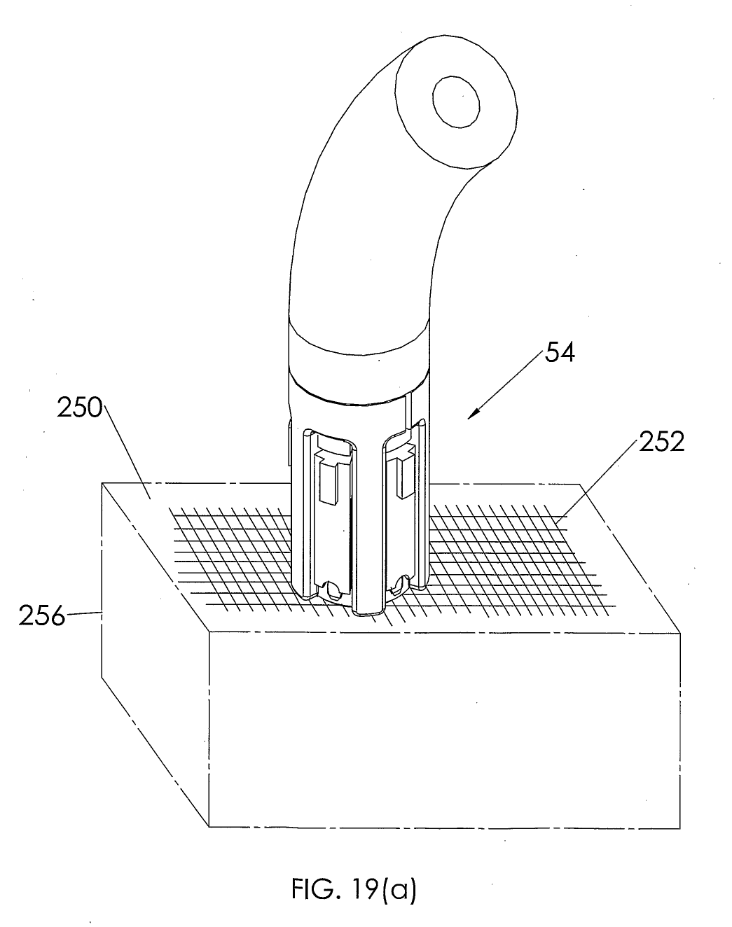

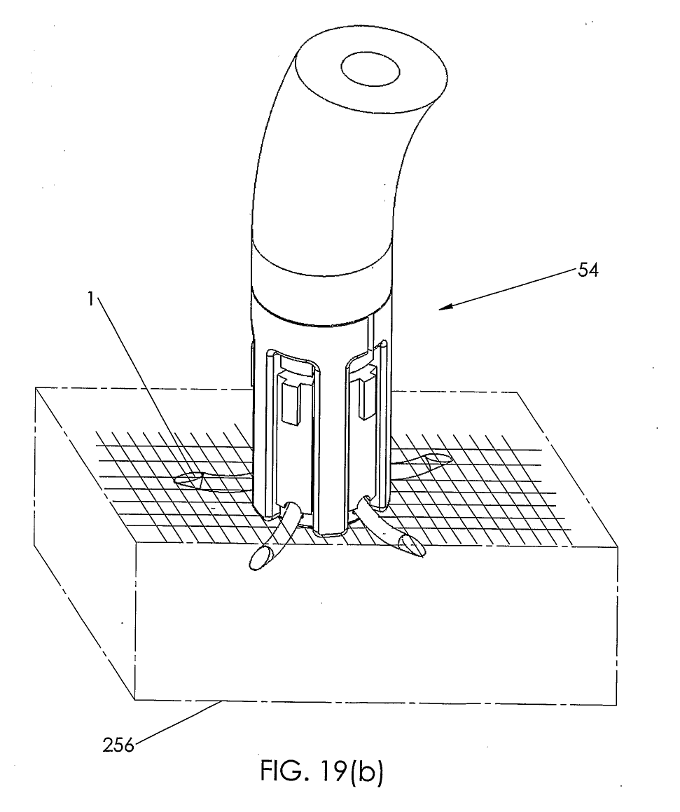

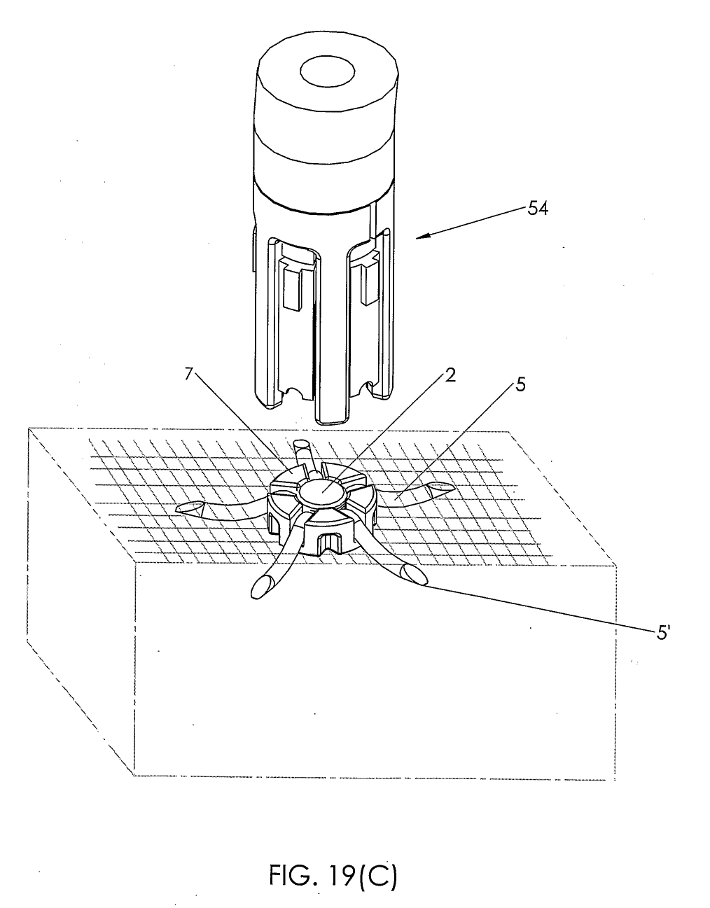

FIG. 19 shows deployment of the fastener 1. In FIG. 19(a), a piece of mesh 252 has been applied to a surface of a body tissue 256, indicated by broken lines in FIG. 19. The tip 54 of the fastening device has been delivered to the tissue surface 250 at a site where the fastener 1 is to be deployed in the body in order to fasten the mesh 252 to the surface 250. The tip 54 has been applied to the mesh 252. Deployment of the fastener occurs by compressing the undeployed fastener to bring the crown 2 towards the baseplate 7. This is accomplished by depressing a trigger 61 towards a handle 62 of the fastening device 60. Squeezing the trigger 61 causes a pusher 58 to slide within a central lumen 59 of the shaft 63 of the fastening device. The pusher 58 urges the crown 2 towards the baseplate 7, while the baseplate 7 is immobilized by the distal projections 56, of the grippers 47 of the fastening device 60. FIG. 19b shows the partially deployed fastener 1. The prongs 5 of the fastener 1 have slid through the slots 8. The tips 5′ of the prongs have penetrated through the mesh 252 into the tissue surface 250 and into the tissue 256. As deployment of the fastener continues, the fastener becomes locked in its deployed configuration by engagement of the rim 11 of the projection 9 of the crown with the rim 12 of the bore 10 of the baseplate that results in a snap fit mechanism. After deployment and fixation to the tissue and locking of the fastener in this deployed configuration additional compression of the deployed fastener will result in its disengagement of the deployed fastener from the distal projections 56 of the grippers 47 and release of the fastener from the fastening device. (FIG. 19c). In the deployed state, the crown 2 and the baseplate 7 remain on the tissue surface 250, and only the prongs 5 have penetrated into the tissue. The fastening device can then be removed from the body.

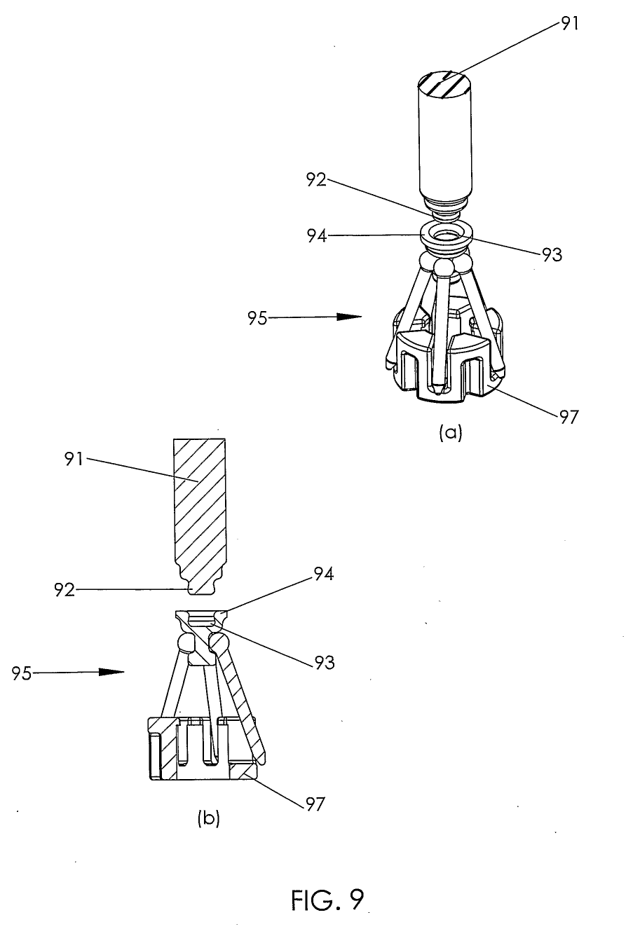

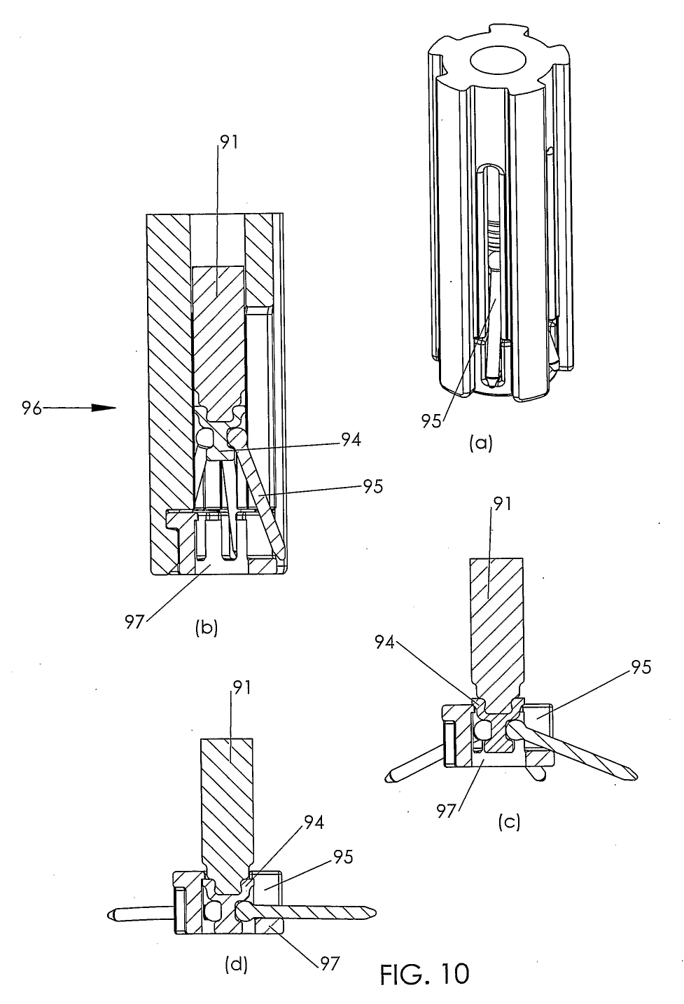

FIG. 9 shows a surgical fastener 95, and the distal end of a pusher 91 of a surgical fastening device in accordance with another embodiment of the invention. The fastener 95 in its undeployed configuration and pusher 91 are shown in a perspective projection in FIG. 9a, and in a longitudinal section in FIG. 9b. In this embodiment, the pusher 91 of the fastening device is provided with a distal laterally protruding rim 92. The rim 92 is configured to engage and firmly grasp the crown 94 of the fastener 95 by snapping into a depression 93 on the upper surface of the crown 94. FIGS. 10a and b show the fastener 95 mounted in the distal end 96 of a fastening device. The pusher 91 is used to urge the crown towards the baseplate 97 while the baseplate 97 is immobilized, as explained above with reference to FIG. 6. This brings the fastener 95 to a deployed configuration shown in FIG. 9c, in which the fastener 95 is not locked. If it is determined that the fastener 95 has not been properly deployed, sliding of the pusher 91 may be reversed, so as to return the fastener 95 to the undeployed configuration shown in FIGS. 9a and 9b. Thus, the deployment of the deployed but unlocked fastener is reversible and the fastener may be extracted from tissue as long as the fastener has not been locked. The extracted fastener may possibly be redeployed at another tissue location. When it has been determined that the fastener has been properly deployed, the pusher is used to snap the crown into the baseplate of the fastener and lock the fastener, as shown in FIG. 10. After locking the fastener and release of the baseplate from the grippers of the device 96 (FIG. 10d), pulling the pusher 91 disengages the pusher 91 from the crown 94 and disengages the fastening device from the fastener. The fastening device may then be removed from the body.

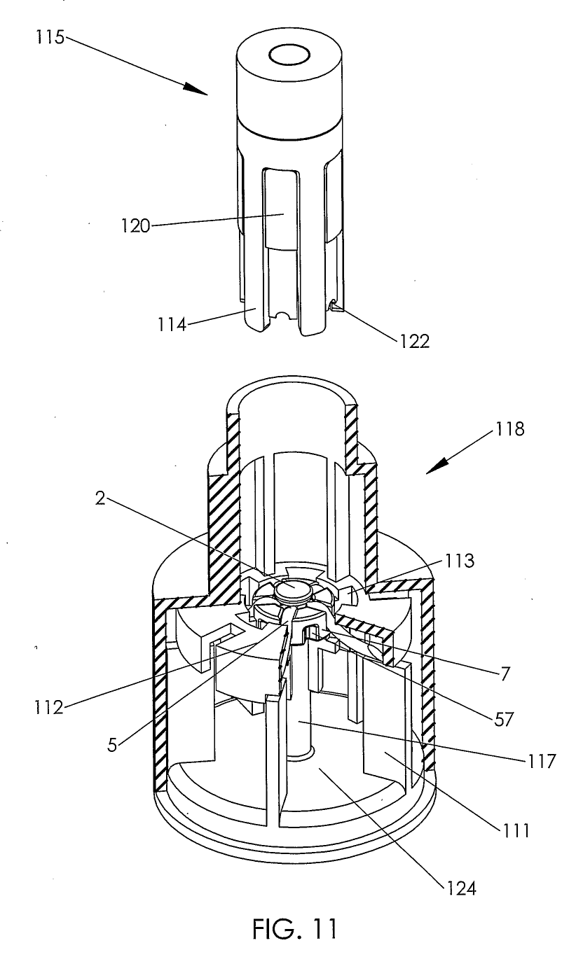

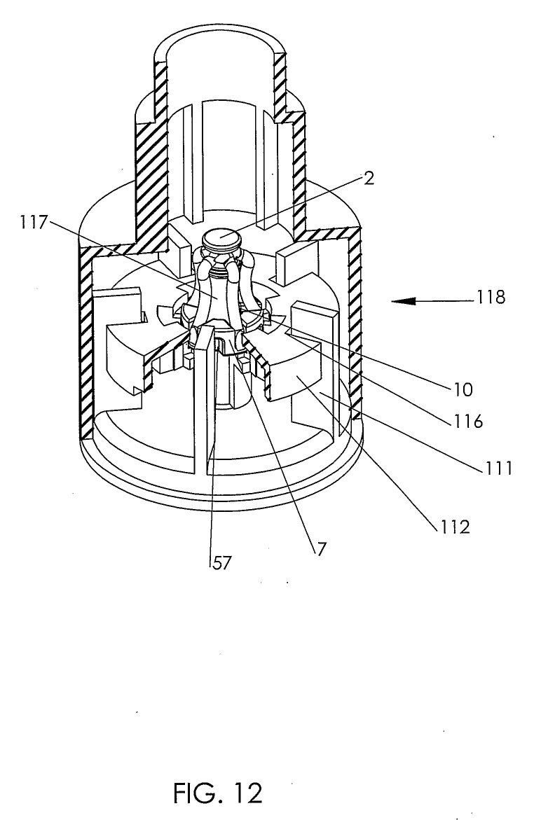

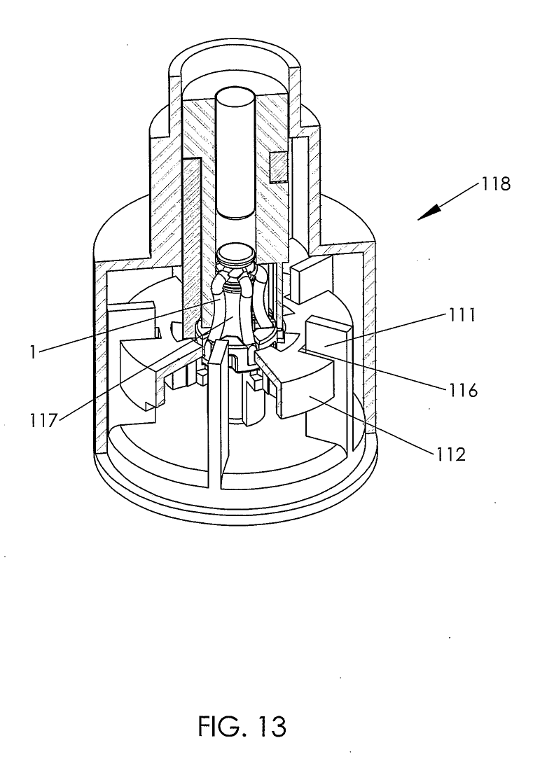

In another embodiment of the invention, shown in FIGS. 11 and 12, a cartridge 118 is be used for mounting the fastener 1 onto the distal end 115 of a fastening device of the invention. The fastener 1 is mounted onto the cartridge in its deployed, but unlocked, configuration. The deployed and unlocked fastener is stable in this state by means of the recesses 8 in the baseplate which stabilize the prongs 5 in the deployed configuration. This cartridge is provided with locking pillars 111 that initially support the baseplate and prevent the fastener from reverting to the undeployed configuration. The cartridge is provided with an unlocking plate 112, that is provided with internal recesses 113 that fit grippers 114 of the distal end 115 of the fastening device and external recesses 116 that fit the locking pillars 111. The cartridge is also provided with a central post 117 upon which the projection 9 of the crown is supported. The post 117 passes through the central bore 10 of the baseplate 7 of the fastener 1. Stabilization means 120 is reversibly or permanently attached to the distal tip 115 of the fastening device between its grippers 114. The distal tip 115 with the attached stabilization means are pushed over the fastener and firmly attached to it by engagement of the projections 122 of the grippers to the corresponding recesses 57 on the baseplate 7. Then the fastening device tip and the attached fastener are rotated until the external recesses 116 of the unlocking plate 112 align with the locking pillars 111 and the baseplate of the fastener is pushed down by the stabilization mean 120 of the fastening device, along the post 117 and the locking pillars to the base 124 of the cartridge. The post 117 of the cartridge prevents the crown 2 from descending with the baseplate, so that the crown and baseplate separate from each other as the fastener to attains its undeployed configuration within the fastening device (FIG. 13). The device tip with the fastener in its undeployed configuration may then be removed from the cartridge. The cartridge may be re used or discarded.

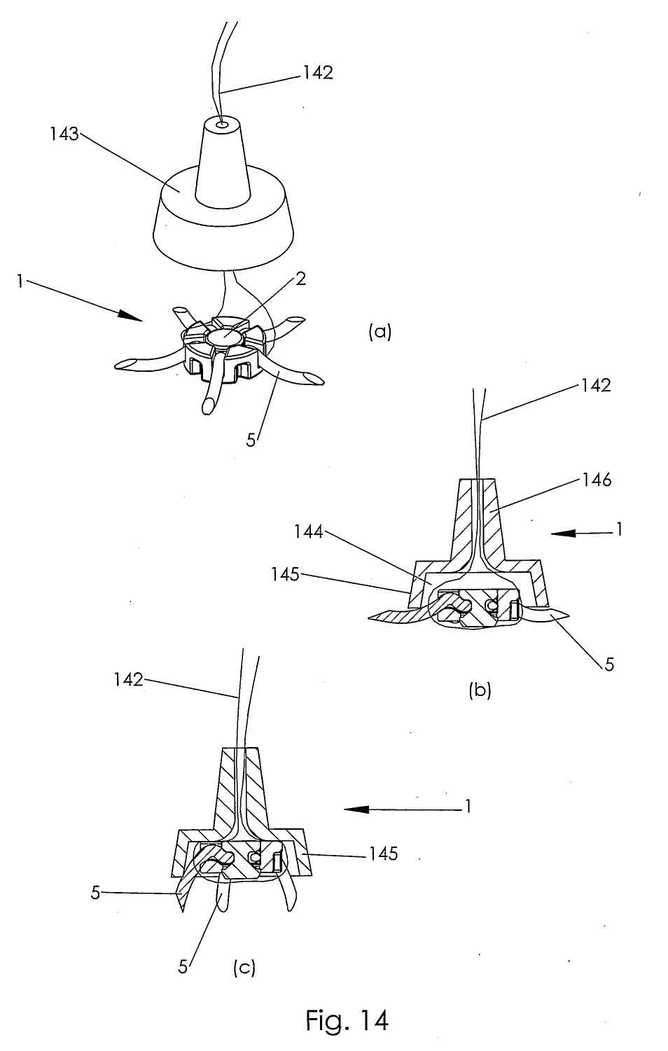

As shown in FIG. 14, a fastener 1 of the invention may be provided with a thread or filament 142 for removal of the fastener, when it is determined that the fastener has not been properly deployed. An extraction device is used having a slender shaft surrounding a central lumen 146 that terminates in a conical tip 143. The filament 142 passes along a lumen of the shaft, exits through the tip 143, passes under the fastener 1, and then returns through the lumen. The tip 143 has an interior space 144 that is configured to contain, partially or completely, the deployed fastener 1, but at least its crown. This depression is provided with a rim 145. In a preferred embodiment the rim 145 of the depression of the extraction device is abutted against the splayed prongs 5 of the deployed fastener. The filament 142 is then pulled while the rim of the conical depression of the extraction device abuts against the prongs 5 (FIG. 14b). As the filament 142 continues to be pulled, the prongs 5 bend bringing the prong tips closer to the axis of the fastener (FIG. 14c), which may then be extracted from the tissue.

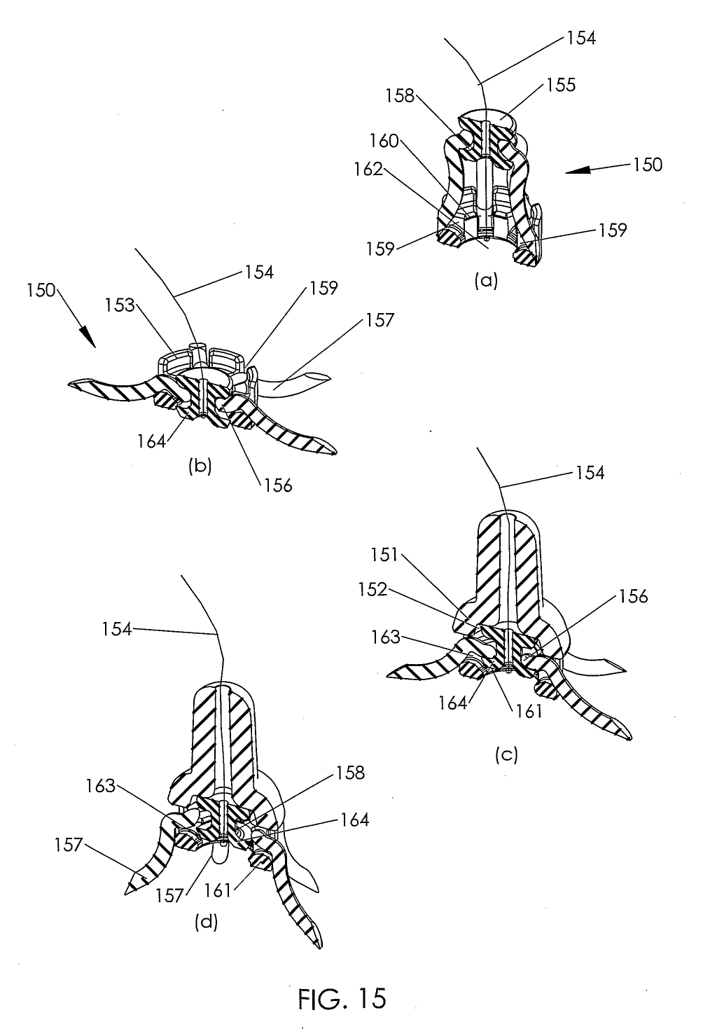

In another embodiment shown in FIG. 15, a fastener 150 has a crown 155 and a baseplate 159. The fastener 150 is shown in its undeployed configuration in FIG. 15a, and in its deployed configuration in FIG. 15b. A rim 160 of the central bore 162 of the baseplate, engages a corresponding rim 163 of the projection 164 of the crown in the deployed configuration and prevents the crown from detaching from the baseplate which would cause the fastener to disintegrate. An extraction device may be used to bring the deployed fastener to its undeployed configuration. A lip 153 surrounds the baseplate 159. A rim 151 of a depression 152 of an extraction device is positioned to abut against the lip 153 of the deployed fastener. By pulling on a filament 154 that in this embodiment, is attached to the crown 155 of the fastener, the crown 155 snaps out and disengages from its locking with the baseplate and is pulled up as it separates from the baseplate (FIG. 15c) which is immobilized by the rim 151. This separation of the crown 155 from the baseplate 159 permits the protuberances 156 of the prongs 157 to rotate in recesses 158 of the crown 155 and partially slide in slots 159 of the baseplate and to partially attain their undeployed configuration (FIG. 15d). As the filament continues to be pulled, the unlocked fastener is removed from the tissue. A combination of bending of the prongs and partial attainment of the undeployed state of the fastener may be employed in additional extraction methods and devices in which the rim of the extraction device depression abuts against the prongs.

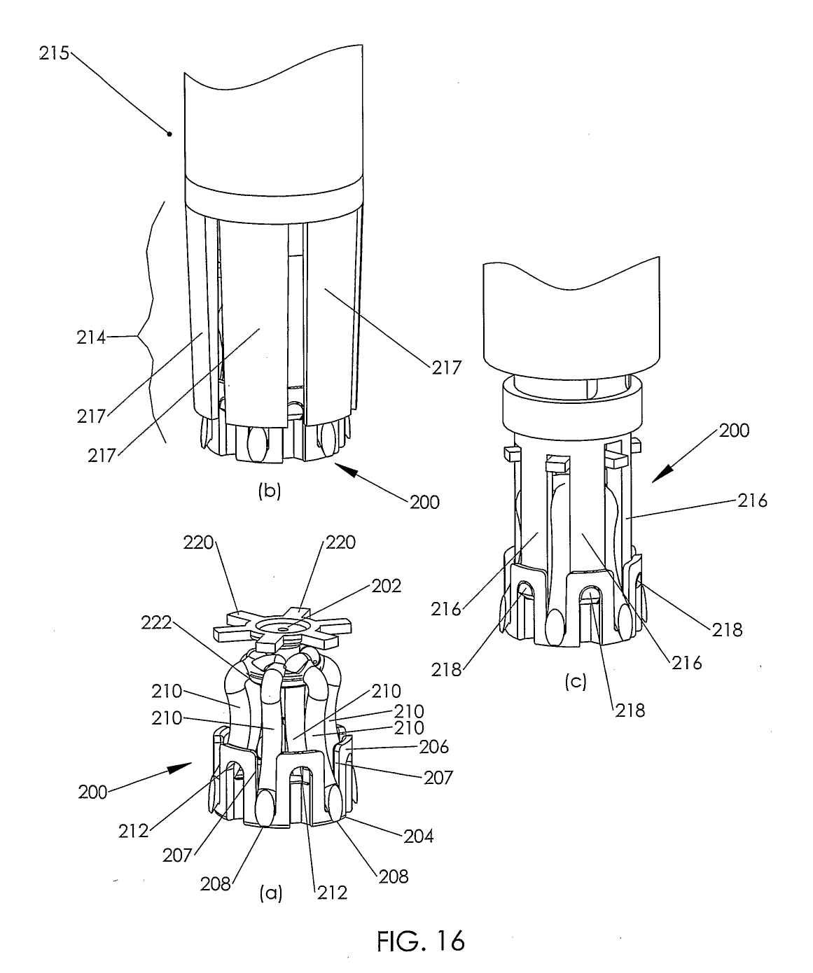

FIG. 16a shows another surgical fastener 200 in accordance with another embodiment of the invention. The fastener 200 is shown in its undeployed configuration in FIG. 16a. The fastener 200 has a crown 202 and a baseplate 204 having a cylindrical lip 206. The cylindrical lip 206 has slots 207 in which the tips 208 of the prongs 210 pass. Between each slot 207 is an arch 212 which is configured to receive a gripper of a fastening device, as explained below.

FIG. 16b shows the fastener 200 after mounting on a stabilization device 214 that has been mounted onto the distal end of a fastening device 215. Panels 217 of the stabilization device are aligned with the arches 212, and partially obstruct the slots 207 and prevent movement of the prongs 210 outwards through the slots 207, in order to prevent movement of the crown 202 towards the baseplate 204 of the of the fastener 200. FIG. 16c shows a view of the stabilization device 214 in which the panels 217 have been removed for the sake of clarity. Grippers 216 in the stabilization device 214 have radially outward facing projections 218 that engage the arches 212 in order to retain the fastener 200 in the distal end of the fastening device. Radially outward extensions 220 extend between the grippers 216 to prevent rotation of the fastener 200 in the stabilization device 214.

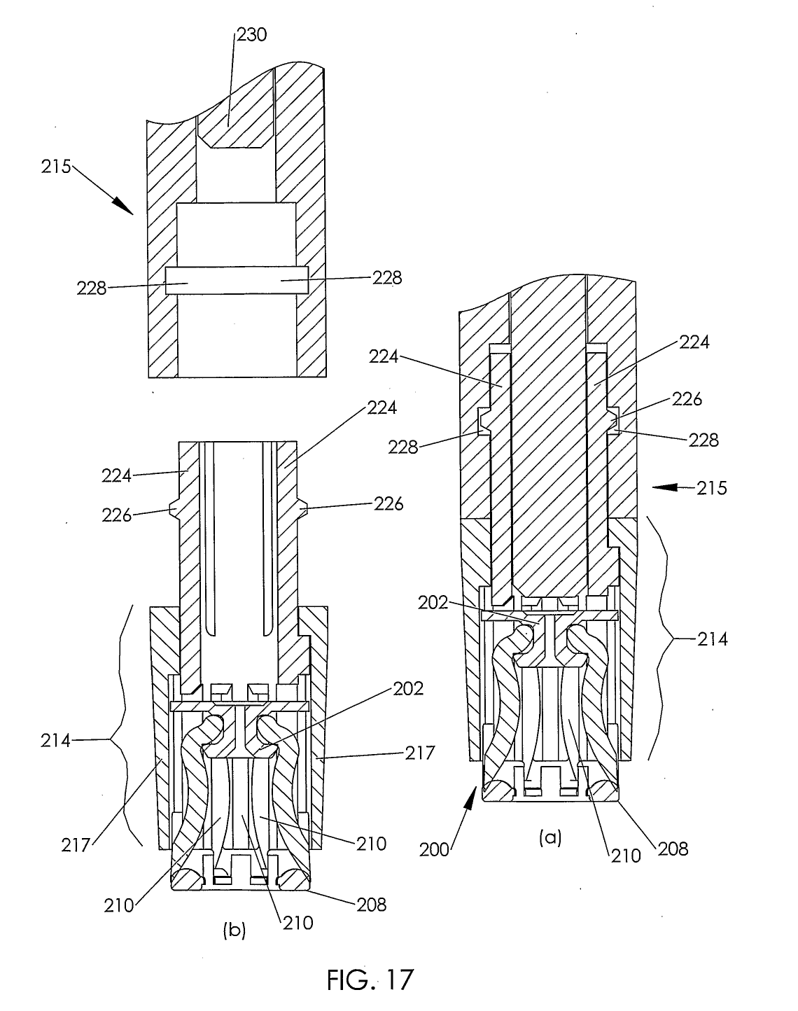

FIG. 17a shows the stabilization device 214 in a longitudinal view. The stabilization device 214 snap fits onto the device 215 by means of a connector 224. The connector has projections 226 that snap fit into an inner circular groove 228 of the fastening device 215.

As explained above in reference to the deployment of the fastener 1, in deployment of the fastener 200, the distal end 214 of the fastening device 215 is delivered to the site of deployment of the fastener 1 in the body. Deployment of the fastener occurs by compressing the undeployed fastener to bring the crown 202 towards the baseplate 204. This is accomplished by causing a pusher 230 to slide within a central lumen of the device 215. The pusher urges the crown 202 towards the baseplate 204, while the baseplate 204 is immobilized by the distal outward projections 218, of the grippers 216. The prongs 210 slightly push the panels 217 outward as the prongs slide in the slots 207. As with the fastener 1, the fastener 200 becomes locked in its deployed configuration by engagement of a rim 222 on the bottom surface of the crown 202 with a rim of a bore (not visible in FIG. 16) of the baseplate that results in a snap fit locking mechanism. After deployment and fixation to the tissue and locking of the fastener in this deployed configuration additional compression of the deployed fastener results in disengagement of the deployed fastener from these distal projections 218 of the grippers 216 and release of the fastener from the fastening device. The fastening device 215, including the empty stabilization device 214, can then be removed from the body. The stabilization device may be detached from the device 215 and possibly discarded. Another stabilization device 214 with a mounted fastener 200 may then be mounted onto the device 215 and the process repeated.

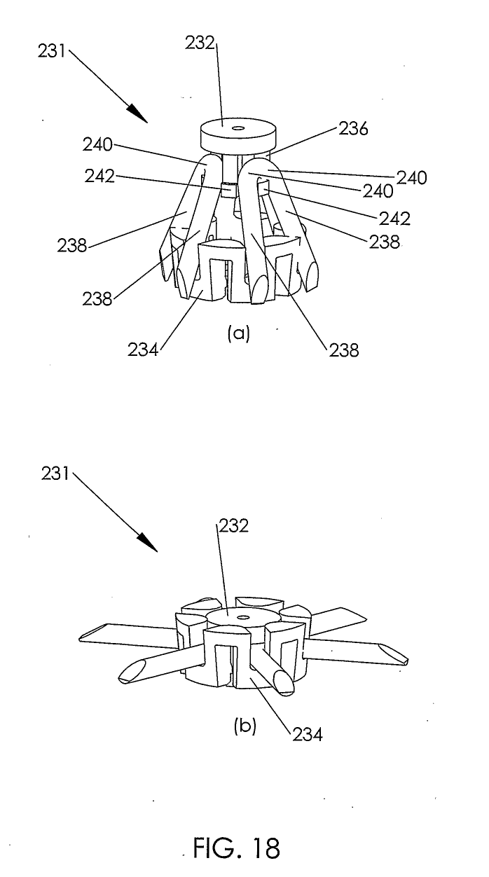

FIG. 18 shows a surgical fastener 231 in accordance with another embodiment of the invention. The fastener 231 is shown in its undeployed configuration in FIG. 18a, and in its deployed configuration in FIG. 18b. The fastener 231 has a crown 232 and a baseplate 234. The fastener 231 has prongs 238. Each prong 238 has a protrusion 240 at its base. The prongs 238 are organized into adjacent pairs, where the protrusions 240 of the prongs of each pair are joined together, so that each pair of prongs forms an integral, U-shaped structure. The U-shaped structure is hooked onto a hook 242 located under the crown 232, so that the protuberances 240 are free to rotate in a groove 236 around the edge of the crown 232. The fastener 231 is deployed using a fastening device, as explained above with reference to the other embodiments.

1. A surgical fastener having an undeployed configuration and a deployed configuration, comprising:
(a) a first element defining an axis of the fastener having one or more depressions around an edge of the first element;
(b) two or more prongs, each prong having a protuberance at one end, the protuberance being received in one of the depressions of the first element and rotatable in the depression, and each prong having a tip at a second end; and
(c) a second element having one or more wells or holes and two or more slots extending from the well or hole to an edge of the second element;
wherein in the undeployed configuration, each prong passes through a slot of the second element and the tip of each prong is at a first distance from the axis; and wherein, in the deployed configuration, each prong passes through the prong's slot of the second element, and the tip of each prong is at a second distance from the axis that is greater than the first distance.

2. The fastener according to claim 1, wherein the protuberance of each prong is shaped to be prevented from passing through the prong's slot.

3. The fastener according to claim 1, wherein the prongs are not attached or affixed to the crown.

4. The surgical fastener according to claim 1, wherein the first element becomes lodged in the second element in the deployed configuration.

5. The surgical fastener according to claim 1, made from a biodegradable material.

6. The surgical fastener according to claim 1, wherein the prongs are not deformed in the deployed configuration of the fastener in comparison to the prongs in the undeployed configuration.

7. The surgical fastener according to claim 1, wherein the protuberances of a pair of adjacent prongs are joined together.

8. A system for surgical fastening comprising:
one or more surgical fasteners having an undeployed configuration and a deployed configuration, the surgical fasteners comprising:
a first element defining an axis of the fastener having one or more depressions around an edge of the first element;
two or more prongs, each prong having a protuberance at one end, the protuberance being received in one of the depressions of the first element and rotatable in the depression, and each prong having a tip at a second end; and
a second element having one or more wells or holes and two or more slots extending from the well or hole to an edge of the second element;
wherein in the undeployed configuration, each prong passes through a slot of the second element and the tip of each prong is at a first distance from the axis; and wherein, in the deployed configuration, each prong passes through the prong's slot of the second element, and the tip of each prong is at a second distance from the axis that is greater than the first distance; and
a fastening device configured to deploy one or more of the surgical fasteners.

9. The system according to claim 8, wherein the fastening device is configured to undeploy a deployed fastener.

10. The system according claim 8, wherein the fastening device comprises a pusher configured to urge the first element of a fastener mounted on the fastening device towards the second element of the fastener.

11. The system according to claim 10, wherein the pusher is configured to attach to the first element of a fastener mounted on the fastening device.

12. The system according to claim 11, wherein the fastening device is configured to move the first element of a fastener mounted on the fastening device away from the second element of the fastener.

13. The system according to claim 8, further comprising one or more stabilization devices for stabilizing one of the surgical fasteners in the undeployed configuration.

14. The system according to claim 13, wherein the fastening device is configured to receive an assembly comprising a stabilization device and a surgical fastener.

15. The system according to claim 14, wherein the assembly is retained in the fastening device by grippers.

16. The system according to claim 14, wherein the assembly is retained in the fastening device by a snap fit.

17. The system according to claim 13, further comprising a cassette configured to hold an assembly comprising the stabilization device and a fastener.

18. The system according to claim 8, further comprising an assembly comprising a stabilization device and a fastener.

19. The system according to claim 8, further comprising a mesh material.

20. A method for surgical fastening, comprising:
providing a system for surgical fastening, the system comprising:
one or more surgical fasteners having an undeployed configuration and a deployed configuration, the surgical fasteners comprising:
a first element defining an axis of the fastener having one or more depressions around an edge of the first element;
two or more prongs, each prong having a protuberance at one end, the protuberance being received in one of the depressions of the first element and rotatable in the depression, and each prong having a tip at a second end; and
a second element having one or more wells or holes and two or more slots extending from the well or hole to an edge of the second element;
wherein in the undeployed configuration, each prong passes through a slot of the second element and the tip of each prong is at a first distance from the axis; and wherein, in the deployed configuration, each prong passes through the prong's slot of the second element, and the tip of each prong is at a second distance from the axis that is greater than the first distance; and
a fastening device configured to deploy one or more of the surgical fasteners;
deliverying at least a portion of the fastening device to a body site where a fastener is to be deployed; and
using the fastening device to deploy fastener at the body site.
